(19) United States
(12) Patent Application Publication
(10) Pub. No.: US 2010/0228295 A1
(43) Pub. Date: Sep. 9, 2010

(54) Variable Radius Vertebra Bend Restrictor

(75) Inventor: William H. Whitefield, Houston, TX (US)

Correspondence Address: CONLEY ROSE, P.C.;David A. Rose, P. O. BOX 3267, HOUSTON, TX, 77253-3267, US

(73) Assignee: Whitefield Plastics, Houston, TX (US)

(21) Appl. No.: 12/400,340

(22) Filed: Mar. 9, 2009

(51) Int. Cl. A61B 17/70; A61B 17/88

(52) U.S. Cl. 606/278; 606/279

(57) ABSTRACT

A bend restrictor for a flexible conduit includes at least two adjacent vertebrae. Each vertebra includes a central passage for the flexible conduit, a ball portion, and a receiver portion configured to receive the ball portion. The ball portion of one adjacent vertebra is disposed within the receiver portion of the other adjacent vertebra. The bend restrictor further includes a vertebra insert disposed between the at least two adjacent vertebrae. The vertebra insert prevents a lock out position between the ball portion and the receiver portion.

### BACKGROUND

Bend restrictors are used to prevent overbending of flexible flow lines, cables, umbilicals, and other conduits that may be damaged if bent beyond a certain radius. One type of bend restrictor used for larger flexible conduits is a vertebra bend restrictor (VBR), which is shown in FIGS. 1A and 1B. FIG. 1A shows a half section of an individual vertebra 101. The vertebra 101 includes a ball portion 10 and a receiver portion 105 at opposing ends. The ball portion 110 is adapted to fit into the receiver portion 105 of an adjacent vertebra. Multiple vertebrae 101 are interlocked end-to-end with their respective ball portions 110 and receiver portions 105 to create a VBR, as shown in FIG. 1B. Assembly of the VBR is performed by clamping the receiver portion 105 of two half sections of each vertebra 101 onto the ball portion 110 of another vertebra 101. The half sections of each vertebra 101 are bolted together or otherwise fastened to form a continuous structure around the flexible conduit 131, which is protected within a central passage 120. The vertebra may be made from various materials depending on the operating conditions. Suitable materials include metals, rubber, and polyurethane.

To limit bending, the ball portion 110 includes a flange 111 that fits within a groove 106 inside the receiver portion 105. The flange 111 has two opposing angled surfaces 112 and 113, which respectively contact surfaces 108 and 107 in groove 106 when at the minimum bend radius (R) or lock out position illustrated in FIG. 1B. In the VBR shown in FIGS. 1A and 1B, surfaces 108 and 107 are perpendicular to the axis of the vertebra i 01. When the respective angled surfaces come into contact, the VBR is locked out and begins to absorb bending loads to protect the flexible conduit inside VBR.

Various dimensions of the vertebra 101 may be adjusted to provide a desired minimum bend radius for a selected size of flexible conduit. One variable is a chord length of each vertebra 101, which is defined by the distance between centers of rotation 130A and 130B. Lengthening the chord increases the minimum bend radius. The relative angle allowed between two adjacent vertebrae 101 is another variable for the minimum bend radius. Increasing the relative angle decreases the minimum bend radius. The relative angle can be adjusted by varying distances and angles between respective surfaces of the ball portion 110 and the receiver portion 105 of the vertebra. Once designed, manufactured, and assembled as a VBR surrounding a flexible conduit, the minimum bend radius is fixed.

### SUMMARY OF INVENTION

In one aspect, the present disclosure relates to a bend restrictor for a flexible conduit. The bend restrictor includes at least two adjacent vertebrae. Each vertebra includes a central passage for the flexible conduit, a ball portion, and a receiver portion configured to receive the ball portion. The ball portion of one adjacent vertebra is disposed within the receiver portion of the other adjacent vertebra. The bend restrictor further includes a vertebra insert disposed between the at least two adjacent vertebrae. The vertebra insert prevents a lock out position between the ball portion and the receiver portion.

In another aspect, the present disclosure relates to a method of varying a minimum bend radius of a vertebra bend restrictor. The method includes disposing a ball portion of a first vertebra in a receiver portion of a second vertebra. The ball portion of the first vertebra and the receiver portion of the second vertebra lock out at a first relative angle between chords of the first vertebra and second vertebra. The method further includes passing a flexible conduit through a central passage of the vertebrae and disposing a vertebra insert between the first vertebra and the second vertebra. The vertebra insert restricts the relative angle between the first vertebra and the second vertebra to a second relative angle smaller than the first relative angle between chords of the first vertebra and second vertebra.

In another aspect, the present disclosure relates to a method of deploying a flexible conduit to an offshore location. The method includes assembling a vertebra bend restrictor around a portion of the flexible conduit. The vertebra bend restrictor comprises at least two adjacent vertebrae and provides a first minimum bend radius. The method further includes spooling the flexible conduit, transporting the spooled flexible conduit to the offshore location, and unspooling the flexible conduit, assembling a vertebra insert between the at least two adjacent vertebrae to increase the minimum bend radius. The vertebra insert reduces a maximum relative angle between chords of the at least two adjacent vertebrae. The method further includes deploying the flexible conduit.

Other aspects and advantages of the invention will be apparent from the following description and the appended claims.

### BRIEF DESCRIPTION OF THE DRAWINGS

FIG. 1A is a half of a vertebra for a vertebra bend restrictor.

FIG. 1B is an assembled vertebra bend restrictor including the vertebra shown in FIG. 1A.

FIG. 2 is a vertebra insert for a vertebra bend restrictor in accordance with one embodiment.

FIG. 3 is a vertebra adapted for use with the insert in FIG. 2 in accordance with one embodiment.

FIG. 4A is an assembled vertebra bend restrictor including the vertebra shown in FIG. 3 in accordance with one embodiment.

FIG. 4B is an assembled vertebra bend restrictor including the vertebra insert shown in FIG. 2 and the vertebra shown in FIG. 3 in accordance with one embodiment.

FIG. 5A is an assembled vertebra bend restrictor including a vertebra insert in accordance with one embodiment.

FIG. 5B is an isometric external view of the assembled vertebra bend restrictor shown in FIG. 5A.

### DETAILED DESCRIPTION

The present disclosure relates to apparatus and methods for varying the minimum bend radius of a VBR.

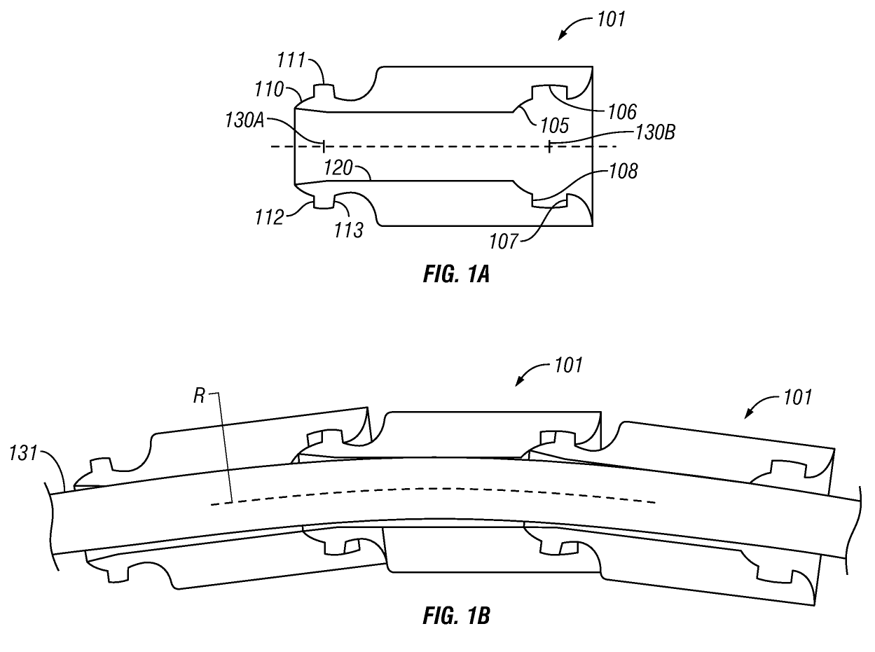

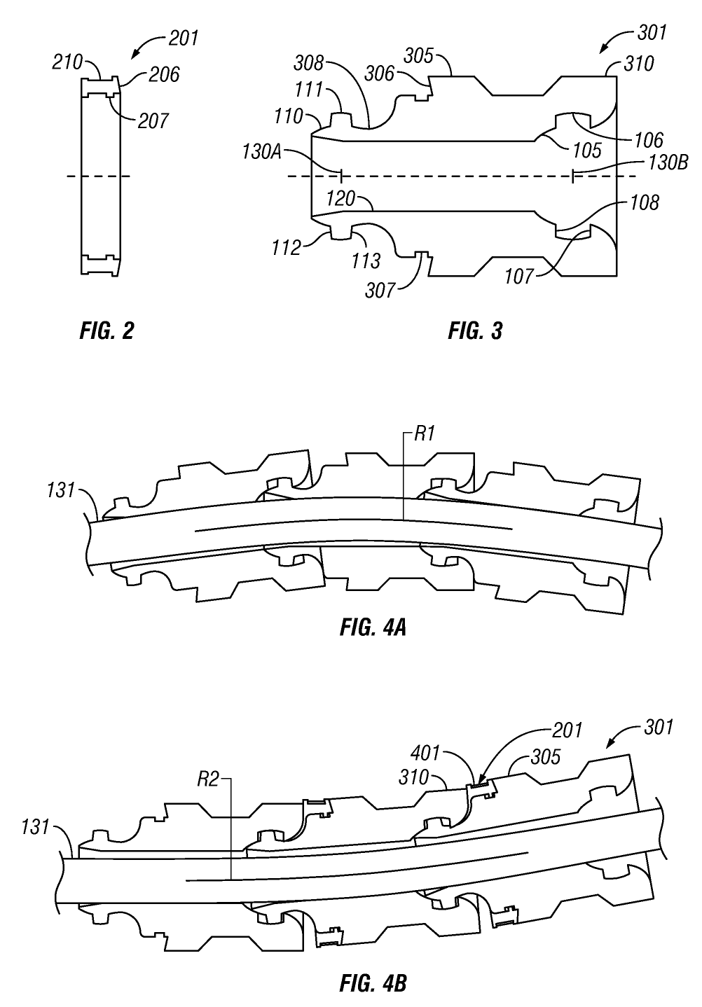

In FIG. 2, a vertebra insert 201 for varying the minimum bend radius of a VBR is shown in accordance with one embodiment. The vertebra insert 201 is adapted to fit between adjacent vertebrae in a VBR to increase the minimum bend radius. The vertebra insert 201 may be formed from two half sections to aid assembly. The material for the vertebra insert 201 may be, for example, metal, plastic, rubber, or polyurethane. A vertebra 301 for use with the vertebra insert 201 is shown in FIG. 3. Like the vertebra 101 shown in FIG. 1A, vertebra 301 includes the ball portion 110 and the receiver portion 105. The vertebra 301 may include an exterior flange 305. The vertebra insert 201 fits between the neck 308 of the ball portion 110 and the exterior flange 305. To improve fit, the vertebra insert 201 and the vertebra 301 may include complimentary surfaces and features. For example, the angle of surface 206 on the vertebra insert may be about the same as the angle of surface 306 on the exterior flange of the vertebra 301. Matching angles between the two complimentary surfaces helps to transfer bending loads between adjacent vertebrae in the VBR. The angle of surface 306 may be negative (less than 90 degrees) to trap the vertebra insert 201 during loading. The vertebra insert 201 may further include an inner flange 207, which fits in insert groove 307 on the vertebra 30l. The complimentary inner flange 207 and insert groove 307 help to keep the vertebra insert 201 in place as the VBR bends and straightens.

FIGS. 4A and 4B show an assembled VBR using the vertebra shown in FIG. 3. FIG. 4A does not include the vertebra insert 201, which provides a minimum bend radius R1. FIG. 4B includes vertebra inserts 201 between adjacent vertebrae 301, which provides an increased minimum bend radius R2. Without the vertebra insert 201, the ball portions 110 and receiver portions 105 interact like the VBR shown in FIG. 1B. Specifically, the angle of the adjacent vertebrae 301 relative to each other is restricted by flange 111 contained inside groove 106. The vertebrae 301 are able to flex until the lock out position is reached between the ball portions 110 and the receiver portions 105, which occurs at minimum bend radius R1 shown in FIG. 4A. In one embodiment, the lock out position maybe at about 9 degrees between chord lines of adjacent vertebrae 301 to provide a minimum bend radius R1 of about 96 inches (2.44 meters) with a chord length of about 12.6 inches (32.0 cm). With 9 degrees bending between each adjacent vertebrae pair, eleven vertebrae 301 (ten adjacent vertebrae pairs) would provide about 90 degrees of bending coverage. Those having ordinary skill in the art will appreciate that chord lengths and relative angles between adjacent vertebrae may be altered according to the design and utilization of any particular flexible conduit.

When an increased minimum bend radius R2 is desired, vertebra inserts 201 are assembled onto each vertebra 301, as shown in FIG. 4B. The vertebra inserts 201 can be added to the VBR without disassembly of any portion of the VBR. Instead, two half sections of the vertebra inserts 201 can be placed around the corresponding exterior portion of each vertebra 301 and secured. In one embodiment, a band 401 is strapped around the vertebra insert 201 to secure the two half sections around the vertebra 301. The vertebra insert 201 may include an exterior groove 210 to aid with placement of the band 401. The band 401 may be metal, such as bands used to secure cargo to pallets. The band 401 allows for quick assembly of the vertebra inserts 201 into the VBR.

The addition of the vertebra inserts 201 changes the loading arrangement of the VBR by shortening the distance between end 310 and exterior flange 305 of adjacent vertebrae 301. As a result, the filly angled lock out position of the ball portions 110 and the receiver portions 105 does not occur because the vertebra insert 201 is compressed between end 310 and exterior flange 305 at a shallower angle between adjacent vertebrae 301. Thus, the vertebra insert 201 effectively reduces the maximum relative angle between adjacent vertebrae 301, which increases the minimum bend radius of the VBR. Continuing with the 96 inches (2.44 meters) minimum bend radius R1 example in FIG. 4A, the minimum bend radius R2 may be increased to about 144 inches (3.66 meters) with the addition of the vertebra inserts 201 between each adjacent vertebrae pair. The increased minimum bend radius R2 is achieved by reducing the maximum relative angle between chords of each adjacent vertebrae pair by about 2.5 degrees. The addition of the vertebra inserts 201 does not affect the chord length of the vertebrae 301.

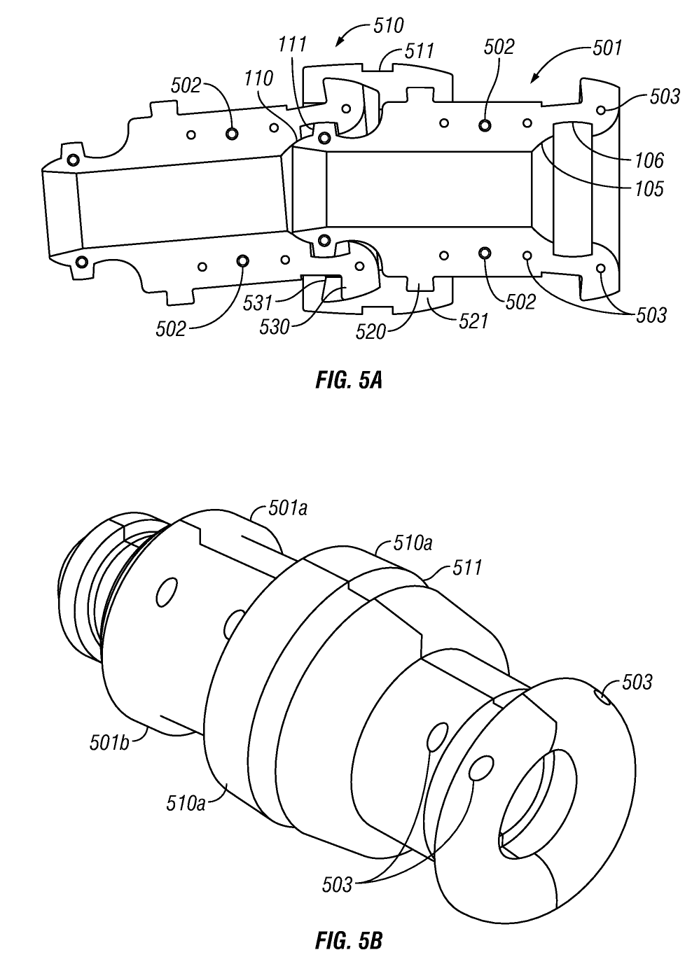

In FIGS. 5A and 5B, an assembled VBR in accordance with another embodiment is shown. As with the vertebra insert 201 in FIG. 2, the addition of a vertebra insert 510 reduces the maximum relative angle between adjacent vertebrae 501, which results in a larger minimum bend radius. Absent the vertebra insert 510, the relative angle between adjacent vertebrae 501 is limited by the lock out position of the ball portion 110 and the receiver portion 105. In this embodiment, the end of the vertebra 501 with the receiver portion 105 includes an exterior ball portion 530. The vertebra insert 510 includes a receiver portion 531, which is adapted to receive the exterior ball portion 530 of the vertebra 501. When the vertebra insert 510 is assembled between adjacent vertebrae 501, the exterior ball portion 530 is received into the receiver portion 531 of the vertebra insert 510. As a result, the maximum relative angle between adjacent vertebrae 501 is restricted by the lock out position of the exterior ball portion 530 and the receiver portion 531, which is at a smaller relative angle than the lock out position between the ball portion 110 and the receiver portion 105.

The vertebra insert 510 and the vertebra 501 may include additional features to improve assembly and aid with force distribution. In one embodiment, the vertebra 501 includes an exterior flange 520, which is adapted to fit in a groove 521 on the vertebra insert 510. The mating flange 520 and groove 521 help to hold the vertebra insert 510 in position during bending, and, at the minimum bend radius, distribute the bending loads between the vertebra insert 510 and adjacent vertebrae 501. The flange 520 may be dovetailed to resist separation during bending. If the flange 520 is dovetailed, assembly of half sections of the vertebra insert 510 may require the use of a mallet or other tool to provide sufficient force to snap the flange 520 into the groove 52 1. For use with a strap or band, the vertebra insert 510 may further include an external groove 511.

The vertebra 501 may be comprised of two half sections 501a and 501b, as shown in FIG. 5B. To aid alignment during assembly, each half section may include alignment pins 502 and/or corresponding holes to receive the alignment pins, as shown in FIG. 5A. The half sections 501 a and 501b may be held together using bolts (not shown) inserted through holes 503 distributed at various locations on the vertebra 501. When a greater minimum bend radius is desired, the vertebra insert 510 is assembled between adjacent vertebrae 501. The vertebra insert 510 may also comprise two half sections 510a and 510b, which may be held together with a band or strap (not shown) in groove 511. When a smaller minimum bend radius is desired, the vertebra insert 510 may be removed by cutting or otherwise removing the band or strap.

The ability to vary the minimum bend radius is useful for VBRs for umbilicals, flying leads, and flexible pipe (collectively referred to as “flexible conduit”) for oilfield applications, especially offshore applications. For transport from a supplier to a service location, flexible conduit is wrapped around a spool. Because of restrictions on the size of the spool for transport on roads, railways, and ships, the smallest minimum bend radius for the flexible conduit is desired. Without additional loading, such as tension, the flexible conduit can withstand a smaller minimum bend radius. Accordingly, the VBR as shown in FIG. 4A may be used. At the point of use for the flexible conduit, other forces may be applied to the flexible conduit as it removed from the spool. For example, in offshore applications, the flexible conduit is exposed to tension from its own weight and any attached equipment, in addition to shear forces from ocean currents and loading from wave action. The additional complex loads cause the flexible conduit to have a greater minimum bend radius to avoid damage. To avoid that damage, the minimum bend radius provided by the VBR can be increased as the flexible conduit is removed from the spool by assembling the vertebra insert, as shown in FIG. 4B. The vertebra inserts may be quickly banded between adjacent vertebrae as the VBR is straightened out while being removed from the spool. The addition of the vertebra inserts requires no disassembly of the VBR already in place. Accordingly, the need to disassemble one VBR to replace it with another VBR with a greater minimum bend radius is avoided. The time savings is particularly valuable in offshore applications in which daily operation costs may be well over a $100,000 per day.

If the flexible conduit is later recovered, the process may be reversed to allow spooling of the flexible conduit. The vertebra inserts may be removed, for example, by cutting off the band. This allows the minimum bend radius of the VBR to be decreased to allow reeling of the flexible conduit onto the spool for transport. As with the original deployment, significant time savings are achieved by avoiding the replacement of one VBR for another VBR to change the minimum bend radius.

Although this detailed description has shown and described illustrative embodiments of the invention, this description contemplates a wide range of modifications, changes, and substitutions. Those having ordinary skill in the art will appreciate that many of the design features shown and described in the above embodiments may be changed or eliminated without departing from the scope of the present disclosure. For example, the vertebra inserts and vertebrae include various complimentary surfaces for improving the fit between each other and for distributing bending loads. Many of the advantages of the present disclosure may be achieved without such features, or with different angles and curves for complimentary surfaces. Accordingly, it is appropriate that readers should construe the appended claims broadly, and in a manner consistent with the scope of the invention.

What is claimed is:

1. A bend restrictor for a flexible conduit, comprising:
at least two adjacent vertebrae, each vertebra comprising,
a central passage for the flexible conduit,
a ball portion, and
a receiver portion configured to receive the ball portion, wherein the ball portion of one adjacent vertebra is disposed within the receiver portion of the other adjacent vertebra; and
a vertebra insert disposed between the at least two adjacent vertebrae, wherein the vertebra insert prevents a lock out position between the ball portion and the receiver portion.

2. The bend restrictor of claim 1, wherein the vertebra insert comprises two half sections.

3. The bend restrictor of claim 2, wherein the bend restrictor further comprises a band wrapped around the two half sections of the vertebra insert.

4. The bend restrictor of claim 3, wherein the vertebra insert comprises an external groove in which the band is disposed.

5. The bend restrictor of claim 1, wherein the at least two adjacent vertebrae comprise external flanges adapted to receive the vertebra insert.

6. The bend restrictor of claim 1, wherein the ball portion of each vertebra comprises a flange received within a groove formed in the receiver portion, wherein sides of the flange on the ball portion do not contact sides of the groove in the receiver portion.

7. The bend restrictor of claim 1, wherein each vertebra comprises an external ball portion proximate to the receiver portion and the vertebra insert comprises a receiver portion configured to receive the external ball portion of the vertebra.

8. A method of varying a minimum bend radius of a vertebra bend restrictor, the method comprising:
disposing a ball portion of a first vertebra in a receiver portion of a second vertebra, wherein the ball portion of the first vertebra and the receiver portion of the second vertebra lock out at a first relative angle between chords of the first vertebra and second vertebra;
passing a flexible conduit through a central passage of the vertebrae;
disposing a vertebra insert between the first vertebra and the second vertebra, wherein the vertebra insert restricts the relative angle between the first vertebra and the second vertebra to a second relative angle smaller than the first relative angle between chords of the first vertebra and second vertebra.

9. The method of claim 8, wherein the vertebra insert comprises two half sections, and wherein the method further comprises:
banding the two half sections of the vertebra insert around an exterior portion of one of the first vertebra and second vertebra.

10. A method of deploying a flexible conduit to an offshore location, the method comprising:
assembling a vertebra bend restrictor around a portion of the flexible conduit, wherein the vertebra bend restrictor comprises at least two adjacent vertebrae and provides a first minimum bend radius;
spooling the flexible conduit;
transporting the spooled flexible conduit to the offshore location;
unspooling the flexible conduit;
assembling a vertebra insert between the at least two adjacent vertebrae to provide a second minimum bend radius, wherein the vertebra insert reduces a maximum relative angle between chords of the at least two adjacent vertebrae; and
deploying the flexible conduit.

11. The method of claim 10, wherein the vertebra insert comprises two half sections, and wherein the method further comprises:
banding the two half sections of the vertebra insert around an exterior portion of one of the first vertebra and second vertebra.

12. The method of claim 10, farther comprising:
recovering the flexible conduit;
removing the vertebra insert; and
re-spooling the flexible conduit.
